(19) United States
(12) Patent Application Publication
(10) Pub. No.: US 2009/0081717 A1
(43) Pub. Date: Mar. 26, 2009

(54) Compositions and Methods For Signal Generation

(76) Inventor: Vijay K. Mahant, Murrieta, CA (US)

Correspondence Address: FISH & ASSOCIATES, PC;ROBERT D. FISH, 2603 Main Street, Suite 1050, Irvine, CA, 92614-6232, US

(21) Appl. No.: 12/212,804

(22) Filed: Sep. 18, 2008

Related U.S. Application Data
(60) Provisional application No. 60/973,408, filed on Sep. 18, 2007.

(51) Int. Cl. C12Q 1/42; C12Q 1/00; C12Q 1/44; C12Q 1/34; C12Q 1/37

(52) U.S. Cl. 435/21; 435/4; 435/19; 435/18; 435/23

(57) ABSTRACT

Improved assay systems comprise a substrate that is modified to include a solubilizing group and/or an at least binary solvent system to provide significantly enhanced signal strength and signal-to-noise ratio. In especially preferred aspects, the assay system is a chromogenic, electrochemical, and/or luminogenic assay system in which an indoxyl-type substrate has a solubilizing group covalently attached to the substrate and/or in which a polar and non-protic solvent is used as a co-solvent.

This application claims the benefit of our copending U.S. provisional patent application with the Ser. No. 60/973,408, which was filed Sep. 18, 2007.

### FIELD OF THE INVENTION

The present invention relates to various compositions and methods for signal generation in biochemical assays, and especially for detectable substrates that allow for multiple detection modes with improved sensitivity and dynamic range.

### BACKGROUND OF THE INVENTION

Signals in biochemical assays can be generated in numerous manners, and depending on the particular assay format, the detectable substances and their respective precursors will have distinct physicochemical properties. For example, where visible signal generation is desired on a membrane (e.g., western blot), precursors will generally be soluble in a developer solution while the detectable product will typically precipitate or otherwise adhere to the membrane. On the other hand, where visible signal generation is desired in a microtiter plate well, the precursor and the detectable product typically remain soluble in the developer solution. Unfortunately, signal strength, sensitivity, and dynamic range are often dictated by quantum efficiency of excitation, quantum yield of fluorescence, the reaction conditions, and optimal signal generation may be prevented by the conditions that a particular sample and/or analyte dictate.

For example, various H₂O₂-driven chemiluminescence reactions (e.g., lucigenin, luminol, etc.) require relatively a high pH (e.g., pH10 and even higher), which is often incompatible with the assay system, resulting in less than desirable sensitivity and/or reproducibility. In another example, and especially where 1,2-dioxetanes are employed as luminogenic agents, one or more stabilizing groups are often required to improve signal strength and test reliability as described in EP 1120652. Alternatively, or additionally, one or more surfactants or polymeric quaternary nitrogen-containing compounds may be included in the developer solution to improve signal generation as taught in U.S. Pat. No. 4,927,769 and WO 94/21821, respectively. In still other examples, indoxyl-type compounds can be used as luminogenic compounds as described in U.S. Pat. No. 5,589,328. Here immediate luminescence may be generated directly upon enzymatic hydrolysis of an indoxyl ester as well as indirectly after decay with concomitant H₂O₂ production. Unfortunately, these substrates often form indigo dyes upon hydrolysis that often adversely affect assays based on quantitative analysis.

Therefore, while numerous signal generating molecules and methods for biochemical assays are known in the art, all or almost all of them suffer from one or more disadvantages. Consequently, there is still a need to provide improved compositions and methods for signal generation in biochemical assays.

### SUMMARY OF THE INVENTION

The present invention is directed to improved biochemical assays in which the substrate is modified with a solubilizing group and/or in which a binary solvent system is employed to thereby increase the specific signal strength and/or the signal-to-noise ratio.

In one aspect of the inventive subject matter, a method of increasing at least one of signal strength and signal-to-noise ratio in an assay system includes a step of providing a substrate having a structure according to Formula I

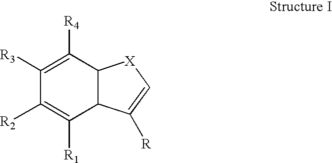

in which X is NR₅, S or O, wherein R₅ is selected from the group consisting of hydrogen, halogen, alkyl, alkoxy, alkoxyalkyl, aryl, aryloxy, hydroxyl, carboxyl, carboxyl alkoxy, aryloxycarbonyl, arylcarbonyl, alkoxy, nitro, acyl, acylamino, and a solubilizing group; R₁, R₂, R₃, and R₄ are independently a radical selected from the group consisting of hydrogen, halogen, haloalkyl, alkylamino, hydroxyl, alkyl, alkoxyalkyl, amino, and a solubilizing group; and in which R is an enzyme-hydrolyzable group. Contemplated methods will include a further step of instructing a user to employ an at least binary solvent system in the assay system where R₁, R₂, R₃, R₄, and R₅ of the substrate are not the solubilizing group.

In especially preferred aspects, R comprises an acyl or acyloxy group, and/or is an ester selected from the group consisting of phosphate, acetate, sulfate, galactopyranoside, glucuronate, glucopyranoside, fructopyranoside, mannopyranoside, and polyhydroxybutyrate. It is further generally preferred that the binary solvent system comprises water as the primary solvent and a polar non-protic solvent as the secondary solvent. Among other suitable solvents in addition to water, dimethyl sulfoxide, dimethylformamide, hexamethylphosphorotriamide, dioxane, a short chain alcohol, formic acid, acetic acid, acetone, anisole, dimethylacetamide, dimethylformamide, pyrrolidone, glycofurol, tetrahydrofuran, benzyl benzoate, an alkyl lactate, ethylhexyl lactate, glycerol, and/or propylene carbonate are especially preferred. While not limiting to the inventive subject matter, it is typically preferred that the non-water solvent in binary solvent systems is present in an amount of at least 2 vol %, and more typically at least 5 vol %.

Viewed from a different perspective, it should therefore be noted that the inventor also contemplates a method of improving performance of a chromogenic or luminogenic assay in which a chromogenic or luminogenic substrate is modified with at least one solubilizing group, and/or in which at least one a co-solvent is included. Such assays may additionally make use of a detergent and/or a polymer that improves the solubility of the chromogenic or luminogenic substrate. Most typically, such assays will be luminogenic assays that employ an indoxyl-type compound (e.g., indoxyl ester) as the signal generating molecule. Moreover, contemplated substrates will preferably include an enzyme-hydrolysable group, and/or the solubilizing group is a sugar, a hydrophilic polymer, or an organic acid. With respect to suitable co-solvents, the same considerations as provided above apply.

Consequently, it should be appreciated that contemplated methods are especially suitable to determine enzymatic activity in a sample (e.g., liquid or tissue sample). Such activity may be determined in numerous manners, including luminometrically and/or spectrophotometrically. In most preferred aspects, the substrate is a luminogenic and/or chromogenic substrate. For example, suitable substrates include those according to Structure I above. Furthermore, it is generally preferred that such contemplated methods will make use of an at least binary solvent system in which the primary solvent is water and in which the secondary solvent is a polar non-protic solvent or other solvent as discussed above.

Various objects, features, aspects and advantages of the present invention will become more apparent from the following detailed description of preferred embodiments of the invention.

### DETAILED DESCRIPTION

The inventor discovered various compounds, compositions, and methods in which one or more luminogenic compounds and/or solvent systems allow for multiple detection modes (e.g., luminescence, color, electrochemical, etc.) and improved dynamic range. In especially preferred aspects, contemplated compounds and methods provide high assay sensitivity and minimize signal quenching, which are critical performance parameters for, inter alia, nucleic acid-based assays, immunoassays, and cell-based assays. More specifically, the inventor has unexpectedly discovered that shortcomings of heretofore known luminogenic, chromogenic, and other systems can be remedied by forming a binary solvent system. Alternatively, or additionally, modified luminogenic compounds with improved solubility in aqueous systems may be used.

In one particularly preferred aspect, a binary solvent system includes water as the primary solvent and a water miscible second solvent as the secondary solvent. As used herein, the term “water miscible” solvent means that the solvent can be mixed with deionized water at neutral pH without phase separation at a concentration of at least 2 vol %, and more typically at least 5 vol %. Of course, it should be recognized that the primary solvent system (i.e., the aqueous system) may include numerous additional components, including buffering agents (e.g., amphoteric agents, acid/base systems, etc.), dissolved salts, detergents (e.g., ionic, zwitterionic, etc.), and polymeric compounds (e.g., charged or neutral), etc. Therefore, typical primary solvents include all known solvents and ingredients currently used in luminogenic reactions.

With respect to the secondary solvent it is generally preferred that the secondary solvent is water miscible in an amount of at least 1 vol %, more preferably at least 2 vol %, even more at least preferably 5 vol %, and most preferably at least 10 vol %. Moreover, it is generally preferred that the secondary solvent is aprotic and even more preferably polar and aprotic. For example, especially preferred polar and aprotic second solvents include dimethyl sulfoxide (DMSO), hexamethylphosphorotriamide (HMPA), dioxane, and dimethylformamide (DMF). Alternatively, polar protic solvents are also considered suitable for use herein and include various short chain alcohols (e.g., C1-C4), formic acid, acetic acid, etc., which may be halogenated where desirable. Additional contemplated solvents include acetone, anisole, dimethylacetamide, tetrahydrofuran, dimethylformamide, pyrrolidone, benzyl benzoate, alkyl lactates (e.g., ethyl lactate, propyl lactate, butyl lactate etc.), glycofurol, ethylhexyl lactate, glycerol, and propylene carbonate. It is still further contemplated that the solvent system may also be a higher system (e.g., ternary or higher) where the third solvent is typically as described for the second solvent above. However, especially preferred third solvents are aprotic and even more preferably polar and aprotic.

Moreover, and where desirable, the binary solvent system (and in some cases the first or second solvent alone) may include additional compounds that modify the solvent system such as to stabilize the educts and/or products, and/or to reduce quenching. For example, suitable solvent systems may include one or more detergents, a polymer (most preferably polar, e.g., PEG) and/or cyclodextrins, etc. Therefore, among other suitable options, cationic and anionic surfactants are preferred detergents, while especially preferred polymers include those with polar side groups (e.g., polyethylene glycols, polyvinyl acetate, etc.).

Most typically, the concentration of the co-solvent (secondary solvent, ternary solvent, etc.) will be in the range of between about 0.1 to 40 vol %, but higher concentrations are also deemed suitable for use herein. For example, where DMSO or DMF are employed as the secondary solvent, the concentration of the secondary solvent is preferably between 1 to 10 vol %. However, other concentrations are also deemed suitable and will typically depend on the type of secondary (and higher) co-solvent. Therefore, suitable concentrations will be in the range of between about 0.01 to 1 vol %, and between about 40 to 80 vol %, and even higher. Similarly, where the binary (or higher) solvent system further comprises a detergent, preferred concentrations of detergents will typically vary between 0.01% (w/w) to 10% (w/w) while polymers (e.g., cyclodextrins) will preferably vary from 1 uM to 100 mM (or between 0.01 wt % to 25 wt % and even higher).

In another particularly preferred aspect, one or more solubilizing groups may be added to the luminogenic, fluorogenic, or chromogenic compound to thereby increase solubility of the substrate, intermediate, detectable product, and/or subsequently formed precipitate. Particularly preferred solubilizing groups include those capable of forming hydrogen bonds with water, polar groups, and/or ionic groups, all of which are preferably covalently coupled to the luminogenic, fluorogenic, or chromogenic compound in one or more positions.

For example, where the compound is a luminogenic indoxyl-type compound, one or more solubilizing groups (e.g., sugars, radicals, sulfate groups, and polyethylene glycol, etc.) may be covalently coupled to the compound as exemplarily depicted in Structure I below

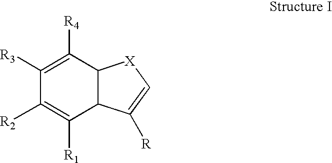

in which X is NR₅, S or O, wherein R₅ is hydrogen, halogen, alkyl, alkoxy, alkoxyalkyl, aryl, aryloxy, hydroxyl, carboxyl, carboxyl lower (C1-C6) alkoxy, aryloxycarbonyl, arylcarbonyl, lower (C1-C6) alkoxy, nitro, acyl, or lower (C1-C6) acylamino groups; wherein R₁, R₂, R₃, and R₄ are independently H, halogen radical, haloalkyl, alkylamino, hydroxyl, alkyl, alkoxyalkyl, amino, in which the alkyl groups contain from 1 to 6 carbons; and wherein at least one of R₁, R₂, R₃, R₄ and R₅ is a solubilizing group. Preferred solubilizing groups are generally relatively small and especially include of polar groups (e.g., hydroxyl, amine, amide, carboxylic acid, sulfonic acid and/or phosphate groups), sugar radicals (e.g., mono or disaccharides), and various polar amino acids (e.g., serine, threonine, etc.). Larger suitable solubilizing groups especially include optionally sulfated or otherwise modified polysaccharides and various polymeric compounds (e.g., polyalkylene oxide, polyalkylene glycol, polyhydroxybutyrates, etc.). R is most preferably a group that can be hydrolytically (or otherwise) cleaved from the compound by one or more enzymes and is therefore preferably an acyloxy group or acyl that can be hydrolyzed by an enzyme of interest. For example, preferred acyl groups are those derived from inorganic acids or small organic acids, and further suitable substituents for R include phosphate, acetate, galactopyranoside, sulfate, glucuronate, glucopyranoside, fructopyranoside, or mannopyranoside radicals, or any of the groups set forth above for R₅.

Additional particularly preferred compounds are described in U.S. Pat. No. 5,589,328, which is incorporated by reference herein. In further contemplated aspects, further luminogenic compounds suitable for use in conjunction with the teachings presented herein also include luminol, various dioxetanes, acridiniumesters, aryl oxalates, and peroxyoxalates, and various substituted anthracenes. Additionally, fluorogenic compounds (e.g., cyanins, various Alexa Fluor dyes, DyLight Fluors, rhodamine, fluorescein, etc.) and numerous chromogenic compounds (e.g., indigo-based) are also contemplated. Of course, each of these alternative compounds may be chemically modified with a solubilizing group, and/or be employed in a binary solvent system as described above.

Regardless of the actual approach taken, it should be recognized that the increased signal strength and increased dynamic range is unexpected as both approaches (use of binary or higher solvent system and/or use of a solubilizing moiety) do not provide phase separation or isolation of a non-aqueous environment that could at least theoretically protect the luminophor from the water environment to so reduce quenching. On the contrary, a person of ordinary skill in the art would expect that the educt, intermediate, and/or product will be present in a more homogenous distribution within the aqueous environment and quenching be more likely to occur. Moreover, as the educt and product quantities are identical in both approaches, the person of ordinary skill in the art would also not expect a reduction in quenching effects by educts, intermediates, and/or products. Without wishing to be bound by any theory or hypothesis, it is contemplated that the secondary (and/or higher) solvent and/or the solubilizing groups will allow for an improved one electron reduction reaction of lucigenin that most likely involves O₂-anionic species, thus possibly reflecting a solvent-solute effect. Alternatively, or additionally, beneficial effects from contemplated systems and compositions may include increased solubility of the educt, intermediate, and/or product in the assay formulation.

In further contemplated aspects, and with particular reference to the above compounds according to Structure I, it should be appreciated that subsequent detection (e.g., luminometric, colorimetric, or electrochemical) may be directly from the indoxyl ester via direct and enzymatic luminescence and/or indirectly by coupling the reaction to an appropriate compound such as lucigenin for enhanced chemiluminescence as shown below (see Scheme A). Further aspects, experiments, and contemplations regarding direct chemiluminescence detection are provided in U.S. Pat. No. 5,589,328, which is incorporated by reference herein. With respect to enhancers for indirect chemiluminescence detection it should be noted that there are various enhancers known in the art, and all of those are deemed suitable for use herein. In such case, the enhancing reaction is believed to involve one electron reduction and may include bis-N-methylacridinium, quinonones, bipyridinium, phenoxazine, phenothiazinium, flavones, tetrazolium, phenols, metals and/or various organometals.

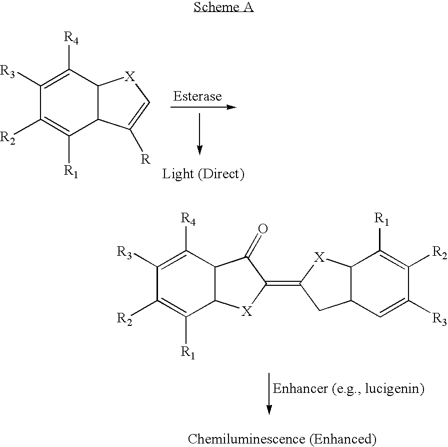

### EXAMPLES

A substrate solution containing 0.8 mg/ml 3-indoxyl phosphate bis(2-amino-2-methyl-1,3-propanediol) salt (Biosynth International) and 0.4 mg/ml lucigenin (Sigma Aldrich) was prepared in 0.1 M Tris buffer, pH 9.0. 10 ul of the sample (blank (0.1 M Tris, pH 9.0 or 1×10⁻¹² moles alkaline phosphatase in 0.1 M Tris, pH 9.0) was added to plastic tubes. 50 ul of the substrate containing 0% DMSO, 0.1% DMSO (v/v), 1% DMSO) (v/v), 10% DMSO (v/v) and 20% DMSO (v/v) was added to respective tubes and water was added to the substrate in appropriate percentage as a control. The reaction was incubated for 45 sec at room temperature and chemiluminescence was measured for 1 sec using an Optocomp I (MGM) luminometer.

TABLE 1

| Substrate | Blank | Alkaline phosphatase |
|---|---|---|
| (vol % DMSO) | (Relative light units) | (Relative light unit) |
| 0 | 472 | 895,068 |
| 0.1 | 511 | 899,207 |
| 1 | 569 | 1,101,369 |
| 10 | 1214 | 1,909,745 |
| 20 | 1917 | 2,375,681 |

As can be clearly taken from the results in Table 1, the quantum yield in relative light units significantly increased with addition of co-solvent. However, it should be noted that at further increasing quantities of co-solvent, the signal-to-noise ratio decreases. While Table 1 depicts only results with DMSO as co-solvent in a binary system using 3-indoxyl phosphate bis(2-amino-2-methyl-1,3-propanediol) salts, similar results are expected with alternative co-solvents in amounts comparable to the above amounts. Furthermore, it should be appreciated that where the substrate is an indoxyl-type compound, detection can be performed in multiple stages. For example, luminescence can be directly and indirectly determined. Additionally, such substrates are also known to form calorimetric indigo-type dyes that can be calorimetrically determined.

Thus, specific embodiments and applications of compositions and methods related to improved signal generation in biochemical assays have been disclosed. It should be apparent, however, to those skilled in the art that many more modifications besides those already described are possible without departing from the inventive concepts herein. The inventive subject matter, therefore, is not to be restricted except in the spirit of the present disclosure. Moreover, in interpreting the specification and contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. A method of increasing at least one of signal strength and signal-to-noise ratio in an assay system comprising:
providing a substrate having a structure according to Formula I 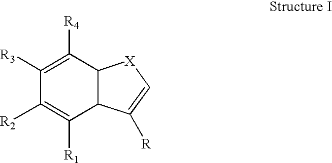
in which
X is NR₅, S or O, wherein R₅ is selected from the group consisting of hydrogen, halogen, alkyl, alkoxy, alkoxyalkyl, aryl, aryloxy, hydroxyl, carboxyl, carboxyl alkoxy, aryloxycarbonyl, arylcarbonyl, alkoxy, nitro, acyl, acylamino, and a solubilizing group;
R₁, R₂, R₃, and R₄ are independently a radical selected from the group consisting of hydrogen, halogen, haloalkyl, alkylamino, hydroxyl, alkyl, alkoxyalkyl, amino, and a solubilizing group;
R is an enzyme-hydrolyzable group; and
instructing a user to employ an at least binary solvent system in the assay system where R₁, R₂, R₃, R₄, and R₅ of the substrate are not the solubilizing group.

2. The method of claim 1 wherein the assay system is a chemiluminescence assay system or a chromogenic assay system.

3. The method of claim 1 wherein R comprises an acyl or acyloxy group.

4. The method of claim 1 wherein R is a radical selected from the group consisting of phosphate, acetate, sulfate, galactopyranoside, glucuronate, glucopyranoside, fructopyranoside, mannopyranoside, and polyhydroxybutyrate, and wherein the enzyme is an ester hydrolyzing enzyme selected from alkaline phosphatase, galactosidase, phosphodiesterase, phospholipase, sulfatase, and protease.

5. The method of claim 1 wherein the binary solvent system comprises water as a primary solvent and a polar non-protic solvent as a secondary solvent.

6. The method of claim 1 wherein the binary solvent system comprises water and a second solvent selected from the group consisting of dimethyl sulfoxide, dimethylformamide, hexamethylphosphorotriamide, dioxane, a short chain alcohol, formic acid, acetic acid, acetone, anisole, dimethylacetamide, dimethylformamide, pyrrolidone, tetrahydrofuran, benzyl benzoate, an alkyl lactate, glycofurol, ethylhexyl lactate, glycerol, and propylene carbonate.

7. The method of claim 1 wherein the binary solvent system comprises water and a second solvent, wherein the second solvent is present in the binary solvent system in an amount of at least 5 vol %.

8. The method of claim 1 further comprising a step of instructing a user to employ an additional solvent where at least one of R₁, R₂, R₃, R₄, and R₅ of the substrate is the solubilizing group.

9. A method of improving performance of a chromogenic, electrochemical, or luminogenic assay, comprising at least one of a step of modifying a chromogenic or luminogenic substrate with at least one solubilizing group, and including at least one a co-solvent, and optionally at least one of a detergent and a polymer to the assay, wherein the polymer improves solubility of the chromogenic or luminogenic substrate.

10. The method of claim 9 wherein the assay is a luminogenic assay.

11. The method of claim 9 wherein the chromogenic or luminogenic substrate comprises an indoxyl ester.

12. The method of claim 9 wherein the solubilizing group comprises a sugar, a hydrophilic polymer, or an organic acid.

13. The method of claim 9 wherein the chromogenic or luminogenic substrate comprises an enzyme-hydrolysable group.

14. The method of claim 9 wherein the improved performance is at least one of increased signal strength and increased signal-to-noise ratio.

15. The method of claim 9 wherein the co-solvent is a polar non-protic solvent.

16. The method of claim 9 wherein the co-solvent is selected from the group consisting of dimethyl sulfoxide, dimethylformamide, hexamethylphosphorotriamide, dioxane, a short chain alcohol, formic acid, acetic acid, acetone, anisole, dimethylacetamide, pyrrolidone, dimethylformamide, tetrahydrofuran, benzyl benzoate, an alkyl lactate, glycofurol, glycerol, ethylhexyl lactate, and propylene carbonate.

17. A method of determining enzyme activity in a sample, comprising:
reacting the sample in a reaction volume with a substrate having a structure according to Formula I 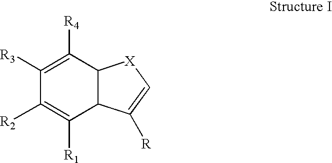
in which
X is NR₅, S or O, wherein R₅ is a radical selected from the group consisting of hydrogen, halogen, alkyl, alkoxy, alkoxyalkyl, aryl, aryloxy, hydroxyl, carboxyl, carboxyl alkoxy, aryloxycarbonyl, arylcarbonyl, alkoxy, nitro, acyl, acylamino, and a solubilizing group;
R₁, R₂, R₃, and R₄ are independently selected from the group consisting of hydrogen, halogen, haloalkyl, alkylamino, hydroxyl, alkyl, alkoxyalkyl, amino, and a solubilizing group;
R is an enzyme-hydrolyzable group; and
wherein the reaction volume comprises an at least binary solvent system where R₁, R₂, R₃, R₄, and R₅ of the substrate are not the solubilizing group.

18. The method of claim 17 wherein the binary solvent system comprises water as a primary solvent and a polar non-protic solvent as a secondary solvent.

19. The method of claim 17 wherein the binary solvent system comprises a solvent selected from the group consisting of dimethyl sulfoxide, dimethylformamide, tetrahydrofuran, hexamethylphosphorotriamide, dioxane, a short chain alcohol, formic acid, acetic acid, acetone, anisole, dimethylacetamide, pyrrolidone, dimethylformamide, benzyl benzoate, an alkyl lactate, glycofurol, glycerol, ethylhexyl lactate, and propylene carbonate.

20. The method of claim 17 wherein the solubilizing group comprises a sugar, a hydrophilic polymer, or an organic acid.
